(19) United States
(12) Patent Application Publication
(10) Pub. No.: US 2026/0199685 A1
(43) Pub. Date: Jul. 16, 2026

(54) IMPLANTABLE STIMULATION DEVICE

(71) Applicants: Imperial College Innovations Limited, London (GB); Chelsea and Westminster Hospital NHS Foundation Trust, London (GB)

(72) Inventors: Fu Siong NG, London (GB); Arunashis SAU, London (GB); Ian WRIGHT, London (GB); Amar AHMED, London (GB)

(21) Appl. No.: 19/128,932

(22) PCT Filed: Nov. 10, 2023
(86) PCT No.: PCT/GB2023/052943

§ 371 Date: May 9, 2025

(30) Foreign Application Priority Data
Nov. 11, 2022 (GB) 2216870.2

(51) Int. Cl. A61N 1/365; A61N 1/362; A61N 1/39

(52) U.S. Cl. CPC A61N 1/365; A61N 1/3621; A61N 1/39622

(57) ABSTRACT

An implantable stimulation device is described. The device comprises signal receiving apparatus for receiving a signal from a heart, and signal delivery apparatus for delivering an output signal to the heart. In response to receiving the signal from the signal receiving apparatus, the implantable stimulation device is configured to determine whether the signal is indicative of ventricular tachycardia. Upon positive determination that the signal is indicative of ventricular tachycardia, it determines whether the signal is indicative of sustained ventricular tachycardia or spontaneously-terminating ventricular tachycardia. Upon positive determination that the signal is indicative of sustained ventricular tachycardia, it sends an output signal via the signal delivery apparatus, else if upon positive determination that the signal is indicative of spontaneously-terminating ventricular tachycardia, it delays sending the output signal via the signal delivery apparatus.

### FIELD

The present invention relates to implantable stimulation devices, in particular, implantable cardioverter defibrillators. The present invention also relates to the configuration of implantable stimulation devices to detect ventricular tachycardia.

### BACKGROUND

Implantable cardioverter defibrillators (ICD) have been used for over 30 years in the management of patients at risk of ventricular arrhythmias (VA) (1). Multiple randomised controlled trials have shown mortality reductions in both primary and secondary prevention settings (2-6).

Implantable cardioverter defibrillator (ICD) therapies have been associated with increased mortality and should be minimised when safe to do so.

There has been a paradigm shift in ICD programming recently, with a move toward avoiding ICD shocks. Initially, rapid detection and treatment of ventricular tachycardia (VT) and ventricular fibrillation (VF) were emphasised, however more recently, there is mounting evidence that ICD shocks can be harmful and should be minimised when safe to do so (7). ICD shocks reduce quality of life (8), may rarely be proarrhythmic (9), and have been associated with excess mortality (10). Potential mechanisms for harm include shock-induced myocardial injury or stunning (11).

### REFERENCES

1. Priori S G, Blomström-Lundqvist C, Mazzanti A, Blom N, Borggrefe M, Camm J, et al. 2015 ESC Guidelines for the management of patients with ventricular arrhythmias and the prevention of sudden cardiac death: The Task Force for the Management of Patients with Ventricular Arrhythmias and the Prevention of Sudden Cardiac Death of the European Society of Cardiology (ESC) Endorsed by: Association for European Paediatric and Congenital Cardiology (AEPC). European Heart Journal. 2015; 36(41):2793-867.
2. Antiarrhythmics versus Implantable Defibrillators I. A comparison of antiarrhythmic-drug therapy with implantable defibrillators in patients resuscitated from near-fatal ventricular arrhythmias. N Engl J Med. 1997; 337(22):1576-83.
3. Bardy G H, Lee K L, Mark D B, Poole J E, Packer D L, Boineau R, et al. Amiodarone or an Implantable Cardioverter-Defibrillator for Congestive Heart Failure. New England Journal of Medicine. 2005; 352(3):225-37.
4. Connolly S J, Gent M, Roberts R S, Dorian P, Roy D, Sheldon R S, et al. Canadian Implantable Defibrillator Study (CIDS). Circulation. 2000; 101(11):1297-302.
5. Kuck K-H, Cappato R, Siebels J, Rüppel R. Randomized Comparison of Antiarrhythmic Drug Therapy With Implantable Defibrillators in Patients Resuscitated From Cardiac Arrest. Circulation. 2000; 102(7):748-54.
6. Moss A J, Zareba W, Hall W J, Klein H, Wilber D J, Cannom D S, et al. Prophylactic Implantation of a Defibrillator in Patients with Myocardial Infarction and Reduced Ejection Fraction. New England Journal of Medicine. 2002; 346(12):877-83.
7. Proietti R, Labos C, Davis M, Thanassoulis G, Santangeli P, Russo V, et al. A systematic review and meta-analysis of the association between implantable cardioverter-defibrillator shocks and long-term mortality. Can J Cardiol. 2015; 31(3):270-7.
8. Carroll D L, Hamilton G A. Quality of life in implanted cardioverter defibrillator recipients: The impact of a device shock. Heart & Lung. 2005; 34(3):169-78.
9. Vollmann D, Lüthje L, Vonhof S, Unterberg C. Inappropriate therapy and fatal proarrhythmia by an implantable cardioverter-defibrillator. Heart Rhythm. 2005; 2(3):307-9.
10. Daubert J P, Zareba W, Cannom D S, Mcnitt S, Rosero S Z, Wang P, et al. Inappropriate Implantable Cardioverter-Defibrillator Shocks in MADIT II. Journal of the American College of Cardiology. 2008; 51(14):1357-65.
11. Hasdemir C, Shah N, Rao A P, Acosta H, Matsudaira K, Neas B R, et al. Analysis of Troponin I Levels After Spontaneous Implantable Cardioverter Defibrillator Shocks. Journal of Cardiovascular Electrophysiology. 2002; 13(2):144-50.
12. Wilkoff B L, Fauchier L, Stiles M K, Morillo C A, Al-Khatib S M, Almendral J, et al. 2015 HRS/EHRA/APHRS/SOLAECE expert consensus statement on optimal implantable cardioverter-defibrillator programming and testing. Heart Rhythm. 2016; 13(2):e50-86.
13. Gasparini M, Menozzi C, Proclemer A, Landolina M, Jacopino S, Carboni A, et al. A simplified biventricular defibrillator with fixed long detection intervals reduces implantable cardioverter defibrillator (ICD) interventions and heart failure hospitalizations in patients with non-ischaemic cardiomyopathy implanted for primary prevention: the RELEVANT [Role of long dEtection window programming in patients with LEft Ventricular dysfunction, Non-ischemic eTiology in primary prevention treated with a biventricular ICD] study. Eur Heart J. 2009; 30(22):2758-67.
14. Gasparini M, Proclemer A, Klersy C, Kloppe A, Lunati M, Ferrer J B, et al. Effect of long-detection interval vs standard-detection interval for implantable cardioverter-defibrillators on antitachycardia pacing and shock delivery: the ADVANCE III randomized clinical trial. JAMA. 2013; 309(18):1903-11.
15. Moss A J, Schuger C, Beck C A, Brown M W, Cannom D S, Daubert J P, et al.

Reduction in inappropriate therapy and mortality through ICD programming. N Engl J Med. 2012; 367(24):2275-83.
16. Saeed M, Hanna I, Robotis D, Styperek R, Polosajian L, Khan A, et al. Programming implantable cardioverter-defibrillators in patients with primary prevention indication to prolong time to first shock: results from the PROVIDE study. J Cardiovasc Electrophysiol. 2014; 25(1):52-9.
17. Wilkoff B L, Williamson B D, Stern R S, Moore S L, Lu F, Lee S W, et al. Strategic programming of detection and therapy parameters in implantable cardioverter-defibrillators reduces shocks in primary prevention patients: results from the PREPARE (Primary Prevention Parameters Evaluation) study. J Am Coll Cardiol. 2008; 52(7):541-50.
18. Koneru J N, Swerdlow C D, Wood M A, Ellenbogen K A. Minimizing inappropriate or “unnecessary” implantable cardioverter-defibrillator shocks: appropriate programming. Circ Arrhythm Electrophysiol. 2011; 4(5):778-90.
19. Volosin K J, Beauregard L-A M, Fabiszewski R, Mattingly H, Waxman H L.

Spontaneous changes in ventricular tachycardia cycle length. Journal of the American College of Cardiology. 1991; 17(2):409-14.
20. Callans D J, Marchlinski F E. Characterization of spontaneous termination of sustained ventricular tachycardia associated with coronary artery disease. The American Journal of Cardiology. 1991; 67(1):50-4.
21. Shaffer F, Ginsberg J P. An Overview of Heart Rate Variability Metrics and Norms. Front Public Health. 2017; 5:258.
22. Ortiz J, Igarashi M, Gonzalez H X, Laurita K, Rudy Y, Waldo A L. Mechanism of spontaneous termination of stable atrial flutter in the canine sterile pericarditis model. Circulation. 1993; 88(4 Pt 1):1866-77.
23. Gary Bih-Fang G, Ellenbogen K A, Wood M A, Stambler B S. Conversion of atrial flutter by ibutilide is associated with increased atrial cycle length variability. Journal of the American College of Cardiology. 1996; 27(5):1083-9.
24. Pogwizd S M, Chung M K, Cain M E. Termination of ventricular tachycardia in the human heart. Insights from three-dimensional mapping of nonsustained and sustained ventricular tachycardias. Circulation. 1997; 95(11):2528-40.
25. Cismaru G, Brembilla-Perrot B, Pauriah M, Zinzius P Y, Sellal J M, Schwartz J, et al. Cycle length characteristics differentiating non-sustained from self-terminating ventricular fibrillation in Brugada syndrome. Europace. 2013; 15(9):1313-8.
26. Frame L H, Simson M B. Oscillations of conduction, action potential duration, and refractoriness. A mechanism for spontaneous termination of reentrant tachycardias. Circulation. 1988; 78(5):1277-87.
27. Frame L H, Rhee E K. Spontaneous termination of reentry after one cycle or short nonsustained runs. Role of oscillations and excess dispersion of refractoriness. Circulation Research. 1991; 68(2):493-502.
28. Conrath C E, Opthof T. Ventricular repolarization: an overview of (patho)physiology, sympathetic effects and genetic aspects. Prog Biophys Mol Biol. 2006; 92(3):269-307.
29. El-Sherif N, Yin H, Caref E B, Restivo M. Electrophysiological mechanisms of spontaneous termination of sustained monomorphic reentrant ventricular tachycardia in the canine postinfarction heart. Circulation. 1996; 93(8):1567-78.
30. Exner Md M, Derek V., Mitchell Md, L. Brent, Wyse Md P D, D. George, Sheldon Md P D, Robert S., Gillis Md, Anne M., Cassidy Bn P, et al. Journal of Interventional Cardiac Electrophysiology. 2000; 4(1):231-9.
31. Sweeney M O, Sherfesee L, DeGroot P J, Wathen M S, Wilkoff B L. Differences in effects of electrical therapy type for ventricular arrhythmias on mortality in implantable cardioverter-defibrillator patients. Heart Rhythm. 2010; 7(3):353-60.
32. Xie J, Weil M H, Sun S, Tang W, Sato Y, Jin X, et al. High-energy defibrillation increases the severity of postresuscitation myocardial dysfunction. Circulation. 1997; 96(2):683-8.
33. Germano J J, Reynolds M, Essebag V, Josephson M E. Frequency and causes of implantable cardioverter-defibrillator therapies: is device therapy proarrhythmic? Am J Cardiol. 2006; 97(8):1255-61.
34. Keene D, Shun-Shin M J, Arnold A D, Howard J P, Lefroy D, Davies D W, et al. Quantification of Electromechanical Coupling to Prevent Inappropriate Implantable Cardioverter-Defibrillator Shocks. JACC Clin Electrophysiol. 2019; 5(6):705-15.
35. Freedman R A, Karagounis L A, Steinberg J S. Effects of sotalol on the signal-averaged electrocardiogram in patients with sustained ventricular tachycardia: relation to suppression of inducibility and changes in tachycardia cycle length. J Am Coll Cardiol. 1992; 20(5):1213-9.

### SUMMARY

According to a first aspect of the invention, there is provided an implantable stimulation device. The device comprises: signal receiving means for receiving a signal from a heart; and signal delivery means for delivering an output signal to the heart. In response to receiving the signal from the signal receiving means, the implantable stimulation device configured to: determine whether the signal is indicative or predictive of ventricular tachycardia. Upon positive determination that the signal is indicative or predictive of ventricular tachycardia, determine whether the signal is indicative or predictive of sustained ventricular tachycardia or spontaneously-terminating ventricular tachycardia; and upon positive determination that the signal is indicative or predictive of sustained ventricular tachycardia, send an output signal via the signal delivery means; else if upon positive determination that the signal is indicative or predictive of spontaneously-terminating ventricular tachycardia, delay sending the output signal via the signal delivery means.

Thus, if the signal from the heart is indicative or predictive of the heart being in a state of sustained ventricular tachycardia, the output signal is sent via the signal delivery means to the heart. If the signal from the heart is indicative or predictive of the heart being in a state of spontaneously-terminating, or self-terminating, ventricular tachycardia, there is a delay in sending the output signal via the signal delivery means to the heart. In this way, sending unnecessary output signals to the heart is avoided. Sending unnecessary output signals to the heart can be harmful as they may reduce quality of life, may rarely be proarrhythmic, and have been associated with excess mortality.

The output signal may be anti-tachycardia pacing (ATP). The output signal may be a shock. The output signal may be ATP followed by a shock, for example, if the ATP is unsuccessful. The ATP may be a series of impulses to pace the heart at a rate faster than the ventricular tachycardia (VT) to terminate the VT. The energy delivered for these impulses may be similar to that used for stimulation of cardiac rhythm. The output signal may be a cardioversion or defibrillation waveform for delivery to the heart.

The output signal (e.g. a shock) may be a single delivery of energy. The output signal may fully depolarise the ventricles and therefore ‘reset’ the cardiac rhythm.

The sustained ventricular tachycardia may be monomorphic sustained ventricular tachycardia.

The delay may be for a pre-determined or pre-programmed time period, for example, 5, 10, 15, 20, 25, or 30 seconds.

A signal indicative or predictive of ventricular tachycardia may be determined by heart rate. To determine whether a signal indicative or predictive of ventricular tachycardia may comprise distinguishing between a signal indicative or predictive of ventricular tachycardia and a signal indicative or predictive of ventricular fibrillation.

The implantable stimulation device may be a transvenous device or a subcutaneous or an extravascular device.

Where the implantable stimulation device is a transvenous device, a signal indicative or predictive of ventricular tachycardia may be determined by: mean magnitude-squared coherence, scatter diagram analysis and/or cross correlation between adjacent unipolar electrograms.

Where the implantable stimulation device is a subcutaneous device or an extravascular device, a signal indicative or predictive of ventricular tachycardia may be determined by time-frequency analysis or cross correlation of two parts of an ECG signal.

The signal receiving means may be a signal receiver for receiving the signal from a heart. The output signal delivery means for delivering an output signal to the heart may be a power source. The signal receiving means may consist of or comprise a one or more leads. The output signal delivery means may consist of or comprise a one or more leads.

Upon positive determination that the signal is indicative or predictive of sustained ventricular tachycardia, the device may be configured to prepare the signal delivery means to send the output signal. The signal delivery means configured to send the output signal may be prepared to be in a state to deliver the output signal within less than 10 seconds, less than 20 seconds, or less than 30 seconds. The time taken to prepare the signal delivery means to send the output signal may depend on the age of the device and/or the age of the battery. The delivery means may comprise a capacitor.

The device may be configured to determine whether the signal is indicative or predictive of spontaneously-terminating ventricular tachycardia; and upon positive determination that the signal is indicative or predictive of spontaneously-terminating ventricular tachycardia, delay sending the output signal via the signal delivery means.

To determine whether the signal is indicative or predictive of ventricular tachycardia the device may be configured to determine whether the signal is indicative or predictive of ventricular tachycardia or ventricular fibrillation.

To determine whether the signal is indicative or predictive of ventricular tachycardia, spontaneously-terminating ventricular tachycardia, sustained ventricular tachycardia, or ventricular fibrillation, the device may be configured to analyse a characteristic of the signal; or analyse the variance of a characteristic of the signal.

The variability of a characteristic of the signal may be analysed and based on the outcome of the analysis, whether the signal is indicative or predictive of ventricular tachycardia, spontaneously-terminating ventricular tachycardia, or sustained ventricular tachycardia may be determined.

If the variability of the characteristic of the signal is below a threshold, this is indicative or predictive of sustained ventricular tachycardia, therefore an output signal is sent via the signal delivery means. If the variability of the characteristic of the signal is above a threshold, this is indicative or predictive of spontaneously-terminating ventricular tachycardia, therefore, sending the output signal via the signal delivery means is delayed. The threshold may be a predetermined threshold.

Other characteristics of the signal may be analysed to determine whether the signal is indicative or predictive of ventricular tachycardia, spontaneously-terminating ventricular tachycardia, sustained ventricular tachycardia, or ventricular fibrillation.

A or the variance of a characteristic of the signal may determine a probability score that the signal is indicative or predictive of sustained ventricular tachycardia, and if the probability score is above a threshold, it may be determined that the signal is indicative or predictive of sustained ventricular tachycardia; else if the probability score is below a threshold, it may be determined that the signal is indicative or predictive of spontaneously-terminating ventricular tachycardia.

The threshold may be dependent on time after the determination that the signal is indicative or predictive of ventricular tachycardia.

For example, between zero and 5 or 10 seconds after the determination that the signal is indicative or predictive of ventricular tachycardia, the threshold is 1, that is, it is not determined that the signal is indicative or predictive of sustained ventricular tachycardia. From 10 seconds after the determination that the signal is indicative or predictive of ventricular tachycardia, the threshold decreases either linearly or non-linearly until the threshold reaches 0.5. The duration of the ventricular tachycardia episode may be 120 seconds or more.

A or the characteristic of the signal may be based on a heartbeat cycle length.

The implantable stimulation device may be configured to measure and/or calculate consecutive heartbeat cycle lengths from the signal from the heart. The implantable stimulation device may be configured to statistically analyse or model one or more characteristics of the signal.

A or the characteristic of the signal may be based on differences between successive heartbeat cycle lengths.

A or the characteristic of the signal may be measured for at least ten repetitions.

The repetitions may be heartbeats, or indicative or predictive of heartbeats.

A or the characteristic of the signal may be based on one or more of mean heartbeat cycle length; heartbeat cycle length standard deviation; root mean square of successive heartbeat RR interval differences; a triangular interpolation of the NN interval histogram; a first order autoregression model coefficient; and a first order autoregression model constant.

The RR interval may be the time elapsed between two successive R-waves of a QRS signal or complex. The NN interval may be a normal-to-normal interval.

The characteristic may be based on one or more of a residual a first order autoregression model. The first order autoregression model may be fitted using ten or more cycle lengths of a heartbeat signal.

A or the characteristic of the signal may be selected based on a random forest classifier to predict the probability of arrhythmia spontaneous termination.

Upon positive determination that the signal is indicative or predictive of spontaneously-terminating ventricular tachycardia, the device may be configured to: monitor the signal to determine whether the signal remains indicative or predictive of spontaneously-terminating ventricular tachycardia or whether the signal is indicative of or predictive sustained ventricular tachycardia; and upon positive determination that the signal is indicative or predictive of sustained ventricular tachycardia, send an output signal via the signal delivery means.

Thus, the implantable stimulation device may be configured to continually monitor the signal from the signal receiving means, and if the signal changes from a signal indicative or predictive of spontaneously-terminating ventricular tachycardia, it sends an output signal to the signal delivery means.

Upon positive determination that the signal is indicative or predictive of spontaneously-terminating ventricular tachycardia, the device may be configured to: monitor the signal to determine whether the signal is indicative or predictive of spontaneously-terminating ventricular tachycardia; and upon positive determination that the signal is indicative or predictive of spontaneously-terminating ventricular tachycardia, continue to delay sending the output signal via the signal delivery means.

Thus, if the signal continues to be indicative or predictive of spontaneously-terminating ventricular tachycardia, the delivery of the output signal may be further delayed as necessary. A first delay period may be applied after the first determination, a second delay period may be applied after a second determination and so on. There may be a cut-off time period where even if the signal continues to be indicative or predictive of spontaneously-terminating ventricular tachycardia, the implantable stimulation device may be configured to send an output signal via the signal delivery means. The cut-off time period may be user-programmable. The cut-off time period may be 30 seconds, 1 minute, 5 minutes, 10 minutes, 15 minutes or 20 minutes.

The signal from the heart may represent the electrical activity of the heart.

The signal received by the signal receiving means may be an electrogram.

The implantable stimulation device may be an implantable cardioverter defibrillator.

The implantable stimulation device may further comprise a generator configured to generate the output signal, and a controller configured to receive the signal from the signal receiving means, determine whether the signal is indicative or predictive of ventricular tachycardia, spontaneously-terminating ventricular tachycardia or sustained ventricular, and upon positive determination, to send a trigger signal to the generator to send the output signal to the signal delivery means.

The implantable stimulation device may further comprise a battery configured to be operatively connectable to the generator. The implantable stimulation device may further comprise a lead connector operatively connected to the signal receiving means.

The controller may be a microcontroller or a field-programmable gate array (FPGA), the controller may be a microprocessor or may be comprised within a microprocessor. The controller may be or be comprised within an application-specific integrated circuit (ASIC).

The controller may comprise one or more of: a ventricular tachycardia detector and analyser; a therapy circuit comprising a pacing circuit and a defibrillation circuit, each operatively connected to the ventricular tachycardia detector and analyser; a therapy controller comprising a pacing controller and a defibrillation controller; wherein the pacing circuit is operatively connected to the pacing controller and the defibrillation circuit is connected to the defibrillation controller; and a controller connected to the detector, pacer, and generator.

The controller may comprise one or more of: a cardiac sensing circuit for sensing electrical signals from the heart; a rate detector for detecting a heart rate from the signal received by the cardiac sensing circuit; and a ventricular tachycardia detector for analysing the heart rate to determine whether the heart rate is indicative or predictive of ventricular tachycardia.

The cardiac sensing circuit for sensing electrical signals from the heart, the rate detector for detecting a heart rate from the signal received by the cardiac sensing circuit, and the ventricular tachycardia detector for analysing the heart rate to determine whether the heart rate is indicative or predictive of ventricular tachycardia may be comprised within the ventricular tachycardia detector and analyser of the controller.

The controller may comprise one or more of: a rate variance analyser; a rate comparator; a cycle length analyser; a heartbeat signal morphology analyser; and a ventricular fibrillation detector.

One or more of the rate variance analyser, rate comparator, a cycle length analyser, heart rate signal morphology analyser, or a ventricular fibrillation detector may be comprised within the ventricular tachycardia detector and analyser of the controller.

The signal receiving means may comprise a signal receiving lead having first and second ends, the first end configured to be implantable to the heart and receive an electrical signal from the heart, and the second end configured to be connectable to the implantable stimulation device.

The signal delivery means may comprise a signal delivery lead having first and second ends, the first end configured to be implantable to the heart and to deliver the output signal to the heart and the second end configured to be connectable to the implantable stimulation device.

The second end of the signal delivery lead and/or the signal receiving lead may be connected to generator of the device.

ATP may be delivered from a right ventricular defibrillator lead of an implantable stimulation device (for example an implantable cardioverter defibrillator (ICD)) and/or the coronary sinus lead (if present). The energy may be delivered in a vector between a first or a first and second coil and the implantable stimulation device generator. The implantable stimulation device defibrillator lead may by transvenous or extravascular.

For transvenous devices, there may be a first coil in the right ventricle, there may be an additional coil in the superior vena cava. For extravascular devices (for example, a subcutaneous or extravascular ICD or an ICD configured to have a substernal defibrillator lead placement) the lead may be configured to be placed under the skin next to the sternum.

The implantable stimulation device may further comprise an external system. The external system may comprise: an external telemetry circuit, a controller, and/or a user interface. The user interface may comprise a presentation device; and a user input device.

Thus, the external system may allow for non-invasive monitoring of the device function, performance, and may allow for patient cardiac history to be downloaded and analysed.

According to a second aspect of the invention, there is provided a method comprising: receiving a signal from a heart; determining whether the signal is indicative or predictive of ventricular tachycardia; upon positive determination that the signal is indicative or predictive of ventricular tachycardia, determining whether the signal is indicative or predictive of sustained ventricular tachycardia or spontaneously-terminating ventricular tachycardia; and upon positive determination that the signal is indicative or predictive of sustained ventricular tachycardia, sending the output signal to the heart; else if upon positive determination that the signal is indicative or predictive of spontaneously-terminating ventricular tachycardia, delaying sending the output signal to the heart.

Determining whether the signal is indicative or predictive of ventricular tachycardia may comprise determining whether the signal is indicative or predictive of ventricular tachycardia or ventricular fibrillation.

Determining whether the signal is indicative or predictive of ventricular tachycardia, spontaneously-terminating ventricular tachycardia, sustained ventricular tachycardia, or ventricular fibrillation may comprise analysing a characteristic of the signal, or analysing the variance of a characteristic of the signal.

A or the characteristic of the signal may be based on one or more of: mean heartbeat cycle length; heartbeat cycle length standard deviation; root mean square of successive heartbeat RR interval differences; a triangular interpolation of the NN interval histogram; a first order autoregression model coefficient and a first order autoregression model constant.

According to a third aspect of the invention, there is provided a computer program which, when executed by at least one processor, causes the at least one processor to perform the method of the second aspect of the invention.

According to a fourth aspect of the invention, there is provided a computer program product comprising a computer-readable medium, which stores the computer program according to the third aspect of the invention.

The method may be implemented in software, e.g. in a (programmable) computer system comprising memory and at least one processor. The method may be implemented, at least in part, in hardware. For example, an application-specific integrated circuit (ASIC) or field-programmable gate array (FPGA) may be used to carry out at least some of the steps of the method.

### BRIEF DESCRIPTION OF THE DRAWINGS

Certain embodiments of the present invention will now be described, by way of example, with reference to the accompanying drawings, in which:

FIG. 1 is a system block diagram of an implantable stimulation device system;

FIG. 2 is a system block diagram of an implantable stimulation device;

FIG. 3 is a system block diagram of a ventricular tachycardia detection and classification module;

FIG. 4 is a system block diagram of an external control system;

FIG. 5 is a process flow diagram to determine whether to send an output signal to delivery means;

FIG. 6 is a plot of inter cycle length internal of consecutive heartbeats for spontaneously-terminating ventricular tachycardia and sustained ventricular tachycardia;

FIG. 7 is a first example of an implantable stimulation device lead;

FIG. 8 is a second example of an implantable stimulation device lead;

FIG. 9 is a plot of the number of equally long normal RR intervals over time;

FIG. 10 is a plot of cycle length measurements from device electrograms;

FIG. 11 is a bar chart of the time for initiation to nadir for spontaneously-terminating ventricular tachycardia and sustained ventricular tachycardia;

FIG. 12 is a bar chart of the percentage change for initiation to nadir for spontaneously-terminating ventricular tachycardia and sustained ventricular tachycardia;

FIG. 13 is a schematic of an electrogram showing an episode of ventricular tachycardia;

FIG. 14 is a Poincare plot of cycle length vs cycle length +1 for the first 10 cycle lengths of each episode;

FIG. 15 is a boxplot of standard deviation for spontaneously-terminating ventricular tachycardia and sustained ventricular tachycardia;

FIG. 16 is a boxplot of triangular interpolation NN intervals for spontaneously-terminating ventricular tachycardia and sustained ventricular tachycardia;

FIG. 17 is a boxplot of regression coefficient for spontaneously-terminating ventricular tachycardia and sustained ventricular tachycardia;

FIG. 18 is a three-dimensional scatter plot of triangular interpolation NN intervals, AR coefficient and standard deviation for spontaneously-terminating ventricular tachycardia and sustained ventricular tachycardia; and

FIG. 19 is a plot of a therapy application threshold for a probability score of sustained ventricular tachycardia over time.

### DETAILED DESCRIPTION OF CERTAIN EMBODIMENTS

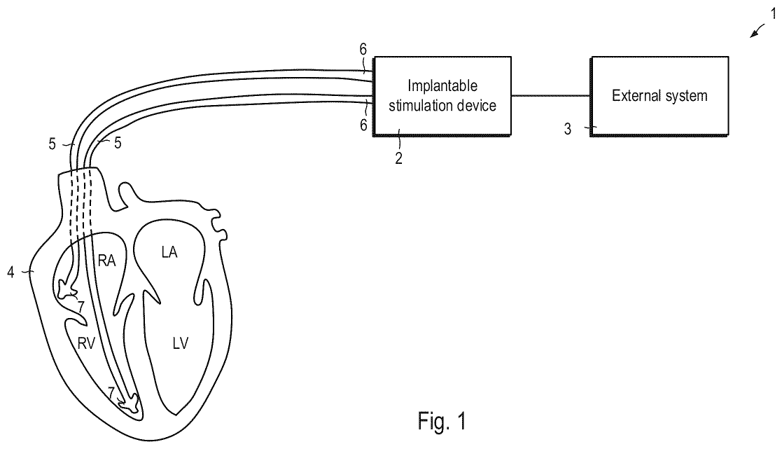

Referring to FIG. 1, an implantable stimulation device system 1 includes an implantable stimulation device 2, for example an implantable cardioverter defibrillator, and an external system 3 which is configured to communicate with the implantable stimulation device 2.

The implantable stimulation device 2 has a signal receiving means and a signal delivery means for receiving and delivering signals to a heart 4. The signal receiving means and signal delivery means may be encompassed or contained within a lead 5 having first and second ends 6, 7, the first end 6 connectable to the implantable stimulation device 2 and the second end 7 connectable to a part of a heart 4. The implantable stimulation device system 1 may also comprise an external system operatively connected to the implantable stimulation device 2 and configured to communicate with or program the implantable stimulation device 2.

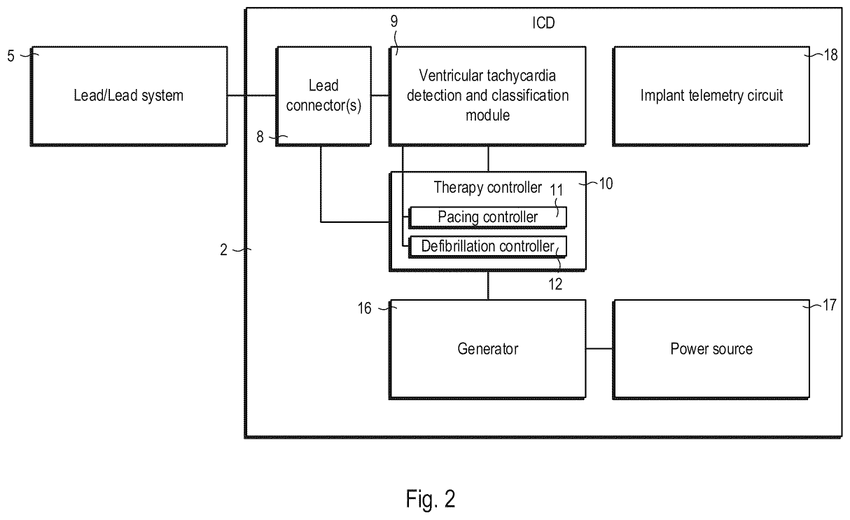

Referring to FIG. 2, the implantable stimulation device 2 includes a lead connector 8 for connecting the second lead end 7 of the lead 5. The implantable stimulation device 2 includes a ventricular tachycardia detection and classification module 9 operatively connected to the lead connectors to allow the module 9 to receive signals from a heart 4. As will be explained in more detail later, the ventricular tachycardia (VT) detection and classification module 9 analyses the signal received from a heart 4 to determine whether the signal is indicative or predictive of ventricular tachycardia rather than, for example ventricular fibrillation, or a healthy heart signal. If the ventricular tachycardia detection and classification module 9 determines that the signal is indicative or predictive of VT, the module 9 may also classify the signal as indicative or predictive of either sustained VT or spontaneously-terminating VT. Identification and classification of the signal may be performed simultaneously. The identification and classification of the signal may be performed as the signal is received. The signal may be indicative or predictive of ongoing VT that will subsequently spontaneously terminate.

The implantable stimulation device 2 further includes a therapy controller 10 which comprises a pacing controller 11 and a defibrillation controller 12. The therapy controller 10 is operatively connected to the VT detection and classification module 9.

Depending on the classification of the signal from the heart, the VT detection and classification module 9 sends a signal to the therapy controller 10 indicating whether a pacing therapy, or a defibrillation therapy is required. Pacing therapy may be, for example, anti-tachycardia pacing (ATP) which is delivered to the heart. Defibrillation therapy may be a signal delivered to the heat which stops the heart. The pacing controller 11 and the defibrillation controller 12 may be operatively connected to additional circuitry such as a pacing circuit (not shown) and a defibrillation circuit (not shown) respectively which may form part of a therapy circuit (not shown). The additional circuitry may generate the signal or part of the signal to be delivered to the heart 4. The implantable stimulation device 2 further includes a generator 16 for generating the signals (e.g. the anti-tachycardia pacing signal and the defibrillation signal) and a power source 17, such as a battery. The therapy controller 10 is operatively connected to the lead connector(s) 8 to provide the signal to the lead 5. The ventricular tachycardia detection and classification module 8 also includes an implant telemetry circuit 18 for receiving information from the external system 3 (FIG. 1). The information received from the external system 3 can be used to program the implantable stimulation device 2, for example, by providing new pacing parameters, therapy delays, and firmware updates.

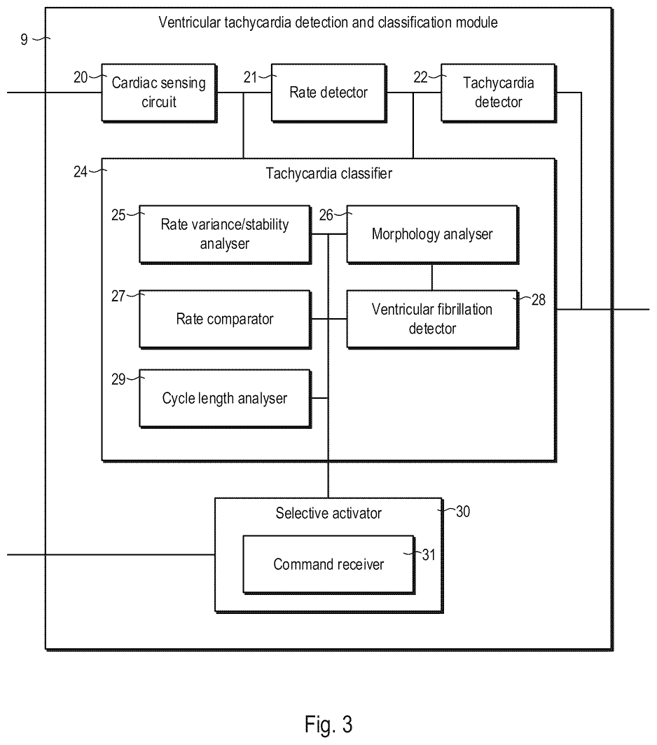

Referring to FIG. 3, the ventricular tachycardia detection and classification module 8 comprises a cardiac sensing circuit 20 for receiving and sensing the signal from the heart 3 via the lead 4 and lead connector 7. That is, the cardiac sensing circuit 20 can analyse the signal received to determine whether the signal is indicative or predictive of a signal from a heart 3. The ventricular tachycardia detection and classification module 8 includes a rate detector 21 which analyses the rate of the signal to determine a heartrate. The ventricular tachycardia detection and classification module 8 includes a tachycardia detector 22 which can analyse the received signal to determine whether the signal is indicative or predictive of ventricular tachycardia.

The ventricular tachycardia detection and classification module 8 includes a tachycardia classifier circuit 24 which includes modules for classifying the received signal into either sustained ventricular tachycardia, spontaneously-terminating ventricular tachycardia (or self-terminating ventricular tachycardia), or in some instances, ventricular fibrillation. These modules include a rate variance or rate stability analyser 25, a morphology analyser 26 for analysing the morphology of the received signal (e.g. the morphology of an electrogram), a rate comparator for comparing rates of a received signal with a predetermined rate, or a threshold rate, a ventricular fibrillation detector 28 dedicated to detect signs of ventricular fibrillation (rather than ventricular tachycardia), and a cycle length analyser 29. The tachycardia classifier 24 is operatively connected to a selective activator 30 comprising a command receiver 31 for receiving the classification of the tachycardia from the tachycardia classifier 24. The selective activator then sends the appropriate command to the therapy controller 9 (FIG. 2), for example if the tachycardia classifier 24 identifies sustained ventricular tachycardia, the selective activator 30 may send a signal to the defibrillation controller 12 (FIG. 2) to send a defibrillation signal (shock) to the heart 4.

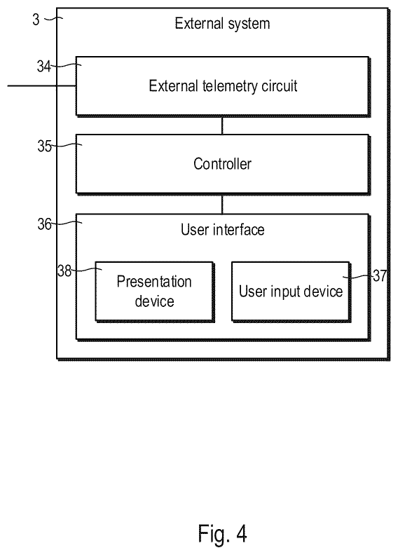

Referring to FIG. 4, the external system 3 includes an external telemetry circuit 34 for communicating with the implant telemetry circuit 18 (FIG. 2), a controller 35, and a user interface 36. The user interface 36 may include a user input device 37 to allow for new parameters or firmware to be input and updated and a presentation device 38 for viewing the inputs.

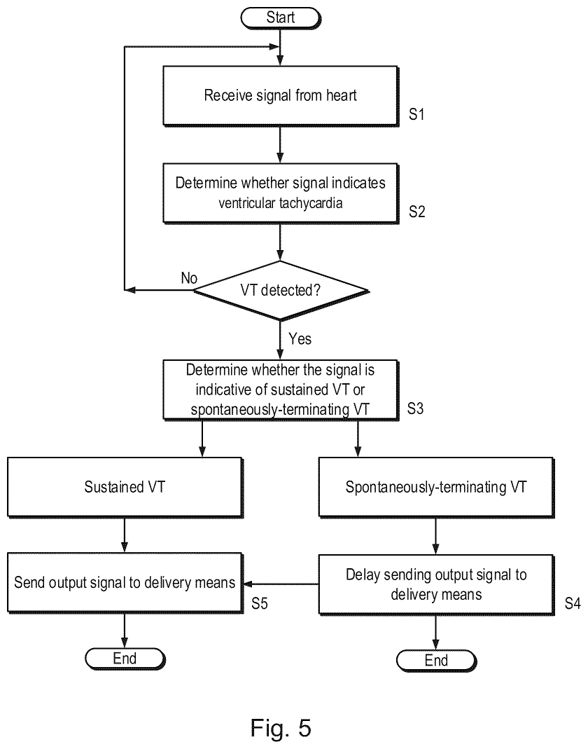

Referring to FIG. 5, a signal from a heart is received via signal receiving means (step S1), the signal is then analysed to determine whether the signal is indicative or predictive of ventricular tachycardia (step S2). If the signal is not indicative or predictive of ventricular tachycardia, the signal continues to be monitored. If the signal is indicative or predictive of ventricular tachycardia, the signal is analysed to determine whether the signal is indicative or predictive of sustained ventricular tachycardia, or spontaneously-terminating ventricular tachycardia (step S3). As will be explained in more detail later, this may be performed by analysing the variance of one or more characteristics of the signal. If spontaneously-terminating ventricular tachycardia is identified, sending an output signal to delivery means (e.g. a pacing signal or a defibrillation signal via the lead 5 to the heart 4) is delayed. The delay may be for a programmable time period (e.g. by programmed by a user using the external system 3), for example by 5, 10, 15, 20, 25, or 30 seconds. Once the delay period has expired, an output signal may be sent to the delivery means (step S5). Alternatively, if sustained ventricular tachycardia is determined, an output signal may be sent to the delivery means immediately (step S5).

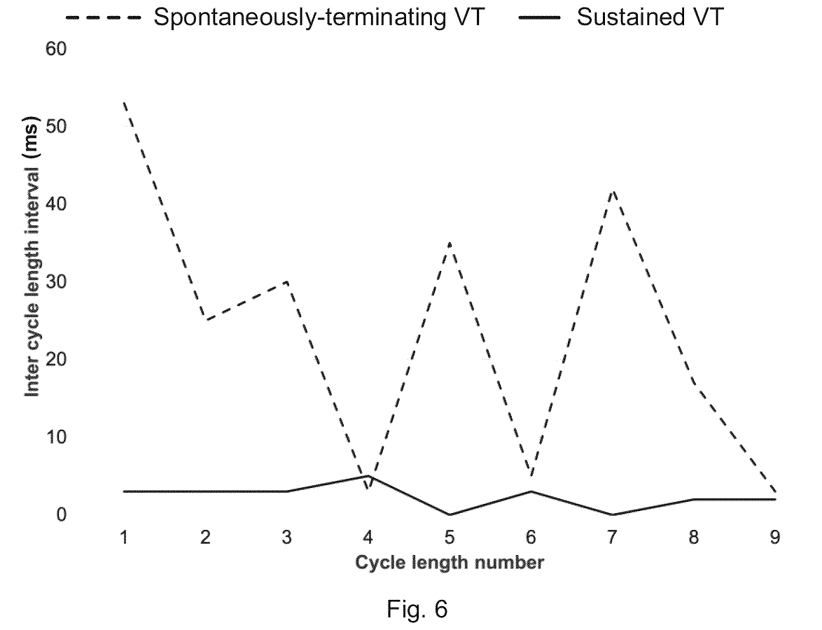

Referring to FIG. 6, a plot of inter cycle length interval in milliseconds over 9 cycle lengths is shown for spontaneously-terminating ventricular tachycardia (dashed line) and sustained ventricular tachycardia (solid line). The spontaneously-terminating ventricular tachycardia inter cycle length interval has a higher mean and variance than the sustained ventricular tachycardia inter cycle length interval.

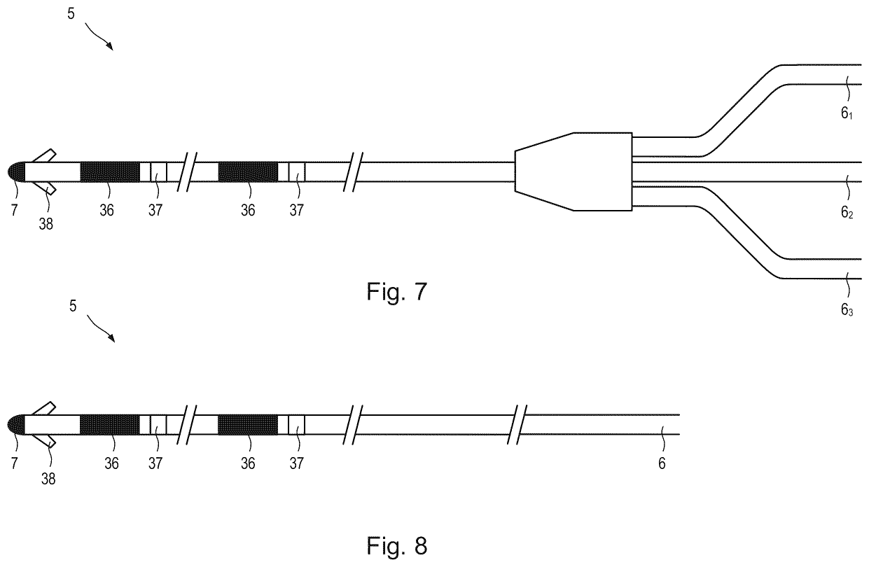

Referring to FIGS. 7 and 8, examples of a lead 5 suitable for connection to a heart 4 having a first end 6₁, 6₂, 6₃ suitable for connecting to the implantable stimulation device 2 and a second end 7 suitable for connecting to, or implanting in a heart 4. The lead may include a coil 36 for providing the output signal to the heart, for example, defibrillation shocks and low- or high-energy cardioversion shocks. The lead 5 may include one or more electrodes 37 for receiving or sensing electrical pulses from the heart and sending them to the implantable stimulation device 2. The lead 5 may include attaching means 38 at or near to the second end 7 such as an anchor or a screw which are suitable for attaching the lead 5 to a suitable part of the heart to receive electrical signals from the heart, and also a part that is suitable for delivering the appropriate therapy to the heart. Referring in particular to FIG. 7, the lead may have more than one first end 6₁, 6₂, 6₃ suitable for connecting to the implantable stimulation device 2. Each first end may have a defined function, such as a defibrillation coil lead end, or a bipolar lead end.

A lead 5 may have at least one distal right ventricular (RV) shock coil 36. A lead 5 may have dual-coils having a second shock coil 36, usually positioned in the superior vena cava (SVC). The leads of FIG. 7 and FIG. 8 may both use a tip electrode 37 as a cathode. If present, a dedicated bipolar lead may have a ring electrode 37 as an anode dedicated to sensing. Alternatively, an integrated bipolar lead may include an RV defibrillation coil, integrated with a shock/signal/pacing circuit, as the anode.

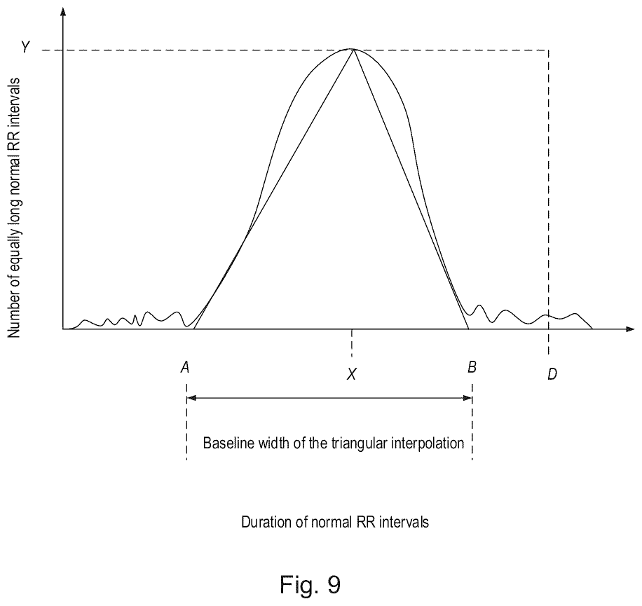

Referring to FIG. 9, a plot of the distribution of the number of equally long normal RR intervals between successive heartbeats against the duration of normal RR intervals is shown. The distribution typically has a normal, or normal-like shape. The baseline width of the triangular interpolation is measured by fitting a triangle to the normal distribution, and measuring the width of the triangle on the x-axis—the duration of normal RR intervals.

The geometric pattern may therefore be interpolated by a mathematically defined shape. For example, the approximation of the distribution histogram by a triangle: a triangular interpolation of the discrete distribution of RR intervals (histogram counts) is used for the TINN [Triangular interpolation of RR (or NN interval) histogram].

### Identification of Spontaneously-Terminating VT Episodes

A potential additional parameter that can be used to identify spontaneously-terminating VT episodes is VT CL stability. Changes in VT CL stability over the duration of a VT episode have been described (19), and VT CL variation has been associated with spontaneous VT termination (20), though the evidence for this are derived predominantly from experiments or from the invasive electrophysiology laboratory setting. We used data from clinical VT episodes stored on ICDs to test our hypothesis that VT CL stability can be used to discriminate between sustained and spontaneously-terminating VT, which may allow an ICD to defer therapy if a VT episode is predicted to be non-sustained.

### Introduction

To reduce ICD shocks, current guidelines recommend longer detection times, allowing some episodes of VT to self-terminate without device therapy (12). The PREPARE, MADIT-RIT, RELEVANT, PROVIDE and ADVANCE III studies (13-17) have all shown the benefits of this approach. Once supraventricular tachycardia (SVT)/VT discrimination algorithms have diagnosed VT, detection and therapy are decided purely on VT cycle length (CL) and number of detection intervals (18).

A potential additional parameter that can be used to identify spontaneously-terminating VT episodes is VT CL stability. Changes in VT CL stability over the duration of a VT episode have been described (19), and VT CL variation has been associated with spontaneous VT termination (20), though the evidence for this are derived predominantly from experiments or from the invasive electrophysiology laboratory setting. We used data from clinical VT episodes stored on ICDs to test our hypothesis that VT CL stability can be used to discriminate between sustained and spontaneously-terminating VT, which may allow an ICD to defer therapy if a VT episode is predicted to be non-sustained.

In use, generally when an implantable stimulation device 2 (e.g. an ICD) delivers a treatment, anti-tachycardia pacing (ATP) would be delivered first followed by a shock if the ATP is unsuccessful. ATP is a series of impulses to pace the heart at a rate faster than the VT to terminate the VT. The energy delivered for these impulses are similar to that used for stimulation of cardiac rhythm. ATP can be delivered from the right ventricular defibrillator lead 5 and/or the coronary sinus lead 5 (if present). The shock may be a single delivery of energy. The energy may be delivered in a vector between one or two coils 36 and the device generator 16. The device defibrillator lead 5 may by transvenous or extravascular. For transvenous devices, there may be one coil in the right ventricle of the heart 4, there may be an additional coil 36 in the superior vena cava. For extravascular devices 2 which may include a substernal defibrillator lead 5 placement, the lead 5 may be located under the skin next to the sternum when in use. The shock acts to fully depolarise the ventricles and therefore ‘reset’ the cardiac rhythm.

### Methods

This was a single-centre retrospective study, approved by the Health Research Authority (Integrated Research Application System ID: 293374, Research Ethics Committee reference: 21/PR/0108).

A review was conducted of the Boston Scientific Latitude Clinician Database to identify all adult patients with Boston Scientific ICDs for any indication, who were followed up at Imperial College Healthcare NHS Trust (London, United Kingdom). Each patient was reviewed for device-detected VT which either self-resolved, required anti-tachycardia pacing (ATP) or an ICD shock. Electrograms (EGMs) were thereafter examined, and episodes were included if a true VT episode lasting longer than 10 CLs was identified. To ensure reliable differentiation between VT and supraventricular tachycardia (SVT), episodes where the atrial rate was equal to, or exceeded, ventricular rates, and episodes from patients without atrial leads were excluded.

### Cycle Length Variability Metrics

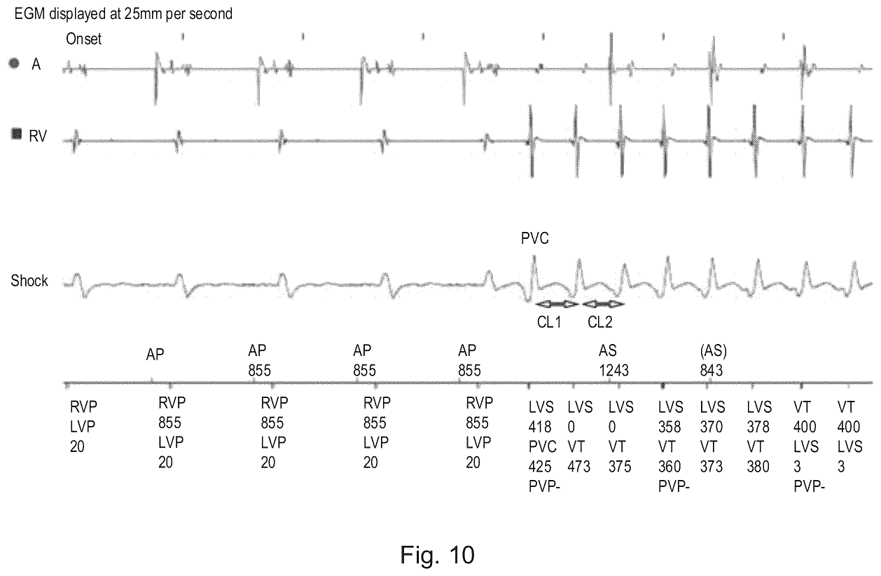

Device-calculated consecutive CLs within the first VT episode were collected. The first VT CL was taken as that following a premature ventricular contraction (PVC) and thus the first CL was recorded as the time between CL 1 of the VT and the preceding PVC (FIG. 10). Where there was uncertainty about the initiation point of the VT, an electrophysiologist was consulted to adjudicate. All included episodes were subsequently divided into two groups; non-sustained (those that self-terminated without therapy) or sustained (those that required ATP or shock). Patients with more than one VT episode could have VT episodes included in both sustained and non-sustained groups of the study.

Referring to FIG. 10, cycle length (CL) measurement(s) from device electrograms: example device electrograms obtained from hospital electronic records of Boston Scientific ICDs. PVC indicated the premature ventricular contraction that induces the VT, CL1 indicates the first VT cycle length, recorded as the difference in time (ms) between the PVC and the subsequent beat, which in this case this is 373 ms. VT CLs for the subsequent beats were taken based on the device measurements (375 ms, 360 ms, 373 ms, etc.) CL: cycle length.

For each VT episode, several descriptive parameters were calculated, including mean of all CL, mean first CL, mean last CL, mean minimum CL, mean episode length, mean number of CLs and mean standard deviation of the CLs. For the initiation-to-nadir analysis 3 features were calculated: time taken to reach nadir (seconds), percent of CL to reach nadir and percent change from first CL to nadir CL.

### Heart Rate Variability Features

To assess the ability to predict sustained VT from stability parameters, several heart rate variability features were calculated. We used the differences between successive CLs to do this (inter-CL intervals). To assess the ability to predict spontaneous termination, we used features derived from the first 10 CLs, as previously described (21). These were: mean, standard deviation (SD), root mean square of successive RR interval differences (RMSSD), NN50, percentage of successive RR intervals that differ by more than 50 ms (pNN50) and the triangular interpolation of the NN interval histogram (TINN). Additionally, a first order autoregression model (AR(1) process) was fitted using the first 10 CLs. The AR coefficient, residual and constant, together with other stability parameters were used as features for a random forest classifier to predict the probability of arrhythmia spontaneous termination. The features with an importance greater than the mean importance of all the features were selected for the final model. The selected features were evaluated using a k-fold cross validation, where k=10.

### Statistical Analysis

Student's t-test was used for parametric data and Mann-Whitney U test all non-parametric data, and considered statistically significant at p<0.05. Analyses were performed using Matlab, Prism 8.0 software (GraphPad Software, California, USA) and Python (version 3.9). Values are expressed as mean±standard error or a median with interquartile range, for parametric and non-parametric data respectively.

### Results

### Patient Characteristics

Four hundred and thirty patients were screened. Twenty-seven patients met the inclusion criteria, from which 69 VT episodes were included. There were 36 spontaneously-terminating VT episodes from 19 patients and 33 sustained VT episodes from 12 patients. Four patients experienced both non-sustained and sustained VT episodes. The median age was 66 (59-75), 22 (81%) were male. 13 patient (48%) had a diagnosis of ischaemic cardiomyopathy. The indication for ICD implantation was for primary prevention in 13 (48%) patients. Further baseline characteristics in the two groups are displayed in Table 1. The median rate for the lowest therapy zone was 185 bpm for spontaneously-terminating VT group and 160 bpm for sustained VT group.

TABLE 1
Baseline characteristics of patients
grouped according to VT outcome.

| | Non-Sustained | | Sustained | | P value |
|---|---|---|---|---|---|
| Number of patients | 19 | | 12 | | |
| Age | 62 | (56-79) | 66 | (58-74) | 0.98 |
| Male | 14 | (73.7) | 11 | (91.7) | 0.36 |
| Smoker | 2 | (16.7) | 1 | (10) | 0.99 |
| Left ventricular ejection | 36 | | 43 | | 0.18 |
| fraction (%) | | | | | |
| First therapy zone | 185 | | 160 | | <0.05 |
| Heart Rate (bpm) | | | | | |
| Indication | | | | | |
| Primary | 11 | (57.9) | 3 | (25.0) | 0.14 |
| Secondary | 8 | (42.1) | 9 | (75.0) | |
| Type | | | | | |
| ICD | 10 | (52.6) | 10 | (83.3) | 0.13 |
| CRT-D | 9 | (47.4) | 2 | (16.7) | |
| Comorbidities | | | | | |
| IHD | 9 | (50) | 7 | (54.3) | 0.72 |
| Diabetes | 7 | (43.8) | 2 | (27.3) | 0.45 |
| AF | 6 | (35.3) | 2 | (16.7) | 0.41 |
| Disease Group | | | | | |
| ICM | 8 | (42.1) | 7 | (58.3) | |
| DCM | 6 | (31.6) | 2 | (16.7) | |
| Idiopathic VT | 2 | (10.5) | 2 | (16.7) | |
| HCM | 1 | (5.3) | 0 | | |
| Sarcoid | 1 | (5.3) | 0 | | |
| ACM | 1 | (5.3) | 1 | (8.3) | |

ICD, implantable cardioverter-defibrillator; CRT-D, cardiac resynchronization therapy defibrillator; IHD, ischaemic heart disease; AF, atrial fibrillation; ICM, ischaemic cardiomyopathy; DCM, dilated cardiomyopathy; VT, ventricular tachycardia; HCM, Hypertrophic cardiomyopathy; ACM, Arrhythmogenic cardiomyopathy.

Sustained VT had shorter first CL and shorter mean CL across the first 10 CLs The mean episode length and number of beats within each episode were longer in the sustained group (episode duration: sustained 11.2±4.4 vs. non-sustained 5.2±1.9 seconds; number of beats: sustained 36±14 vs. non-sustained 16±5 beats, Table 2, p<0.0001). The mean first CL was shorter in the sustained group (329.9±40 vs. 357.9±41 ms, p<0.05). The mean CL across the first 10 CLs was significantly lower in the sustained group (321.6±64 vs 344.1±36 ms, p=0.01). The mean CL across the episode trended towards being shorter in the sustained group, but was not statistically significant.

TABLE 2
Mean ± SD of VT episode descriptors

| | Non-Sustained | Sustained | P value |
|---|---|---|---|
| Number of Episodes (no.) | 36 | 33 | |
| Mean First CL (ms) | 357.9 ± 41 | 329.8 ± 33 | <0.05 |
| Mean Minimum CL (ms) | 312.3 ± 33 | 305.7 ± 33 | 0.47 |
| Mean Episode Duration (s) | 5.2 ± 1.9 | 11.2 ± 4.4 | <0.001 |
| Mean number of beats (no.) | 16 ± 5 | 36 ± 14 | <0.001 |
| Mean Minimum CL across | 317.6 ± 32 | 306.7 ± 32 | 0.16 |
| first 10 CLs | | | |
| Mean CL across first 10 CLs | 344.1 ± 36 | 321.6 ± 64 | 0.01 |
| Mean 10^{th} CL | 337.4 ± 38 | 325 ± 38 | 0.18 |

Sustained VT episodes had significantly lower mean first CL, mean CL across first 10 CLs and longer mean episode length and number of beats. CL: cycle length, VT: ventricular tachycardia

Sustained VT Episodes Reach Nadir CL Faster than Non-Sustained

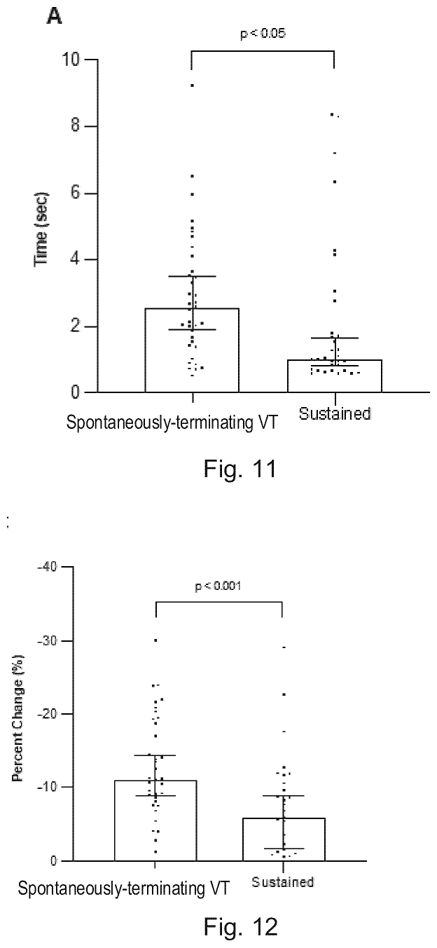

As shown in FIG. 11, the sustained episodes reached their nadir CL within a median of 1.04 seconds (interquartile range 0.7-2.18 s) compared to 2.55 seconds (interquartile range 1.54-3.77 s) in the non-sustained episodes (p<0.05). Within the sustained episodes, the mean percentage decrease from initiation to minimum CL was significantly lower compared to non-sustained episodes (6.0±29% vs 11±30%) (p<0.001). Referring to FIGS. 11 and 12, initiation-to-Nadir Analysis: For sustained VT, compared to spontaneously-terminating VT, there was a (FIG. 11) shorter time to reach nadir CL (shortest VT CL of the episode) and (FIG. 12) reduced percentage change from first CL to nadir CL. Episodes of spontaneously-terminating VT n=36, sustained VT n=33.

Spontaneously-Terminating VT Episodes have More CL Variability

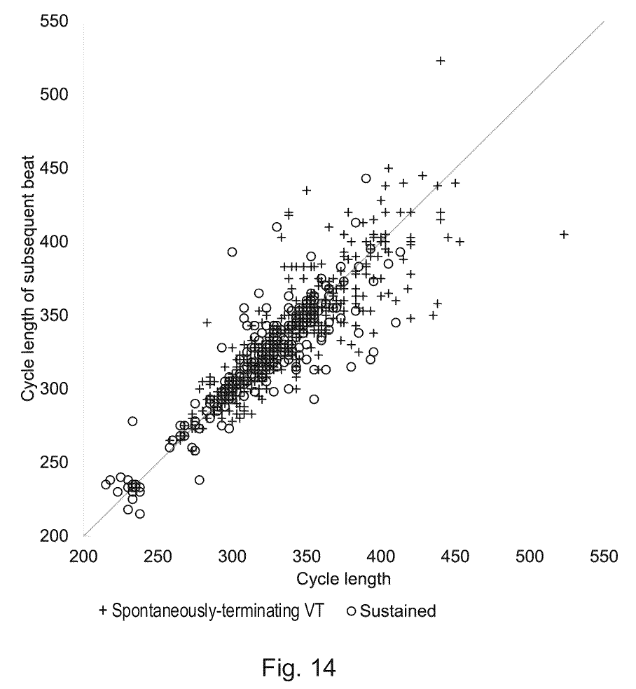

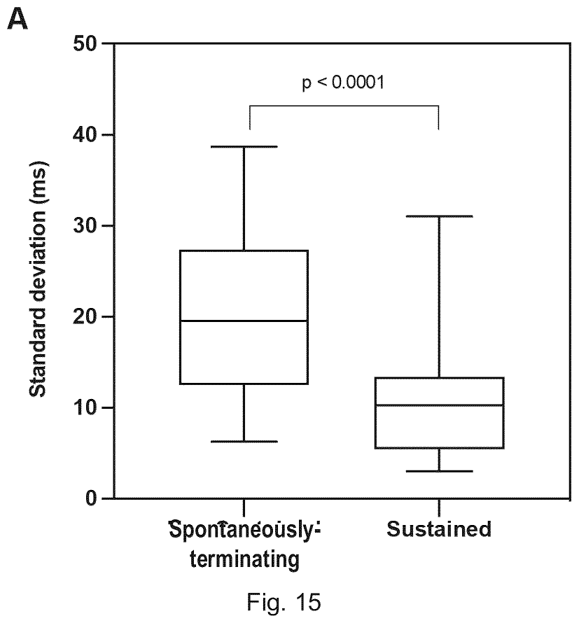

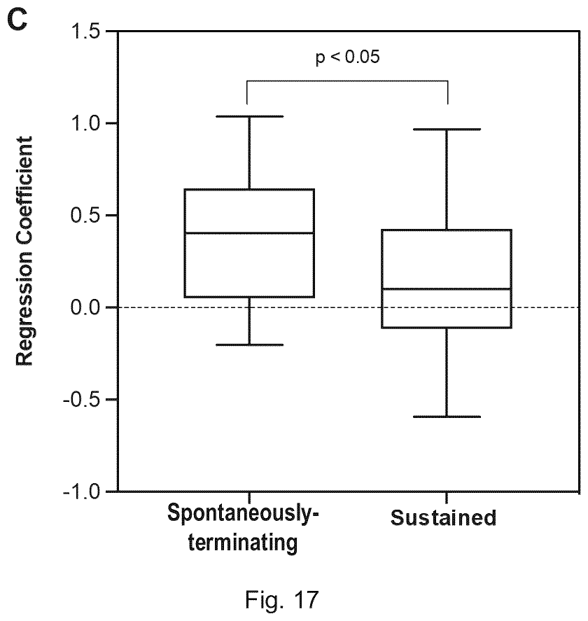

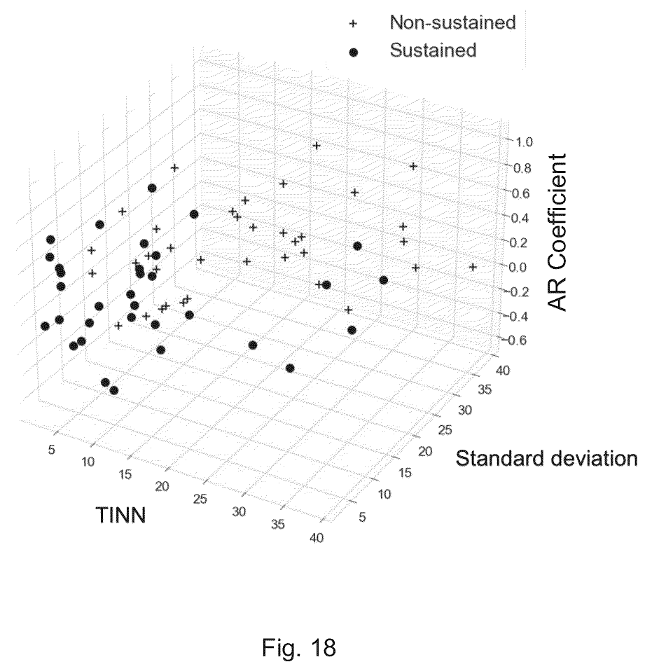

Across the entire episode, the mean of the standard deviation of successive CLs, a measure of CL variability within each episode, was greater in the non-sustained episodes (21.3±9.6 vs 10.6±5.3 ms; p<0.0001). Including only the first 10 CLs of each VT episode, FIG. 14 shows the Poincaré plot of VT CL against the previous VT CL, showing the increased CL variability in non-sustained episodes. When considering just the first 10 CLs of each VT episode, several CL variability parameters were calculated. As shown in FIGS. 15, 16, and 17, mean standard deviation (SD) and TINN, were greater in the non-sustained episodes when compared to sustained VT episodes; (mean SD 20.1±8.9 vs 11.5±7.8 ms, p<0.0001, mean TINN 18.6±8.5 vs 11.1±8.0 ms, p<0.001). Additionally, a first order autoregression model was fitted to each 10-CL series. The autoregression coefficient was significantly greater in non-sustained episodes (mean autoregression coefficient 0.39±0.32 vs 0.14±0.39, p<0.005, FIG. 17). FIG. 18 shows a 3D scatter plot of three features, with partial separation of non-sustained and sustained VT data points.

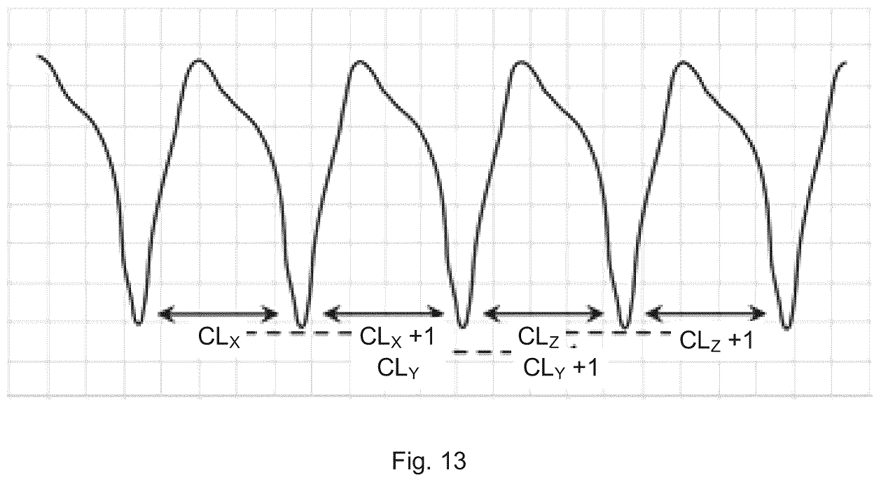

Referring to FIGS. 13, spontaneously-terminating VT episodes showed increased beat-to-beat CL variation in the first 10 CLs: (FIG. 13) Schematic showing an episode of VT. Each CL is paired (dashed line) with the subsequent CL (CL+1). Referring to FIG. 14, a Poincaré plot of CL vs CL+1 for the first 10 CLs of each episode were plotted (total of 9 CL pairs per episode). This showed greater variability for the non-sustained episodes (blue dots), with greater deviation from the grey line (CL=CL+1). Episodes of spontaneously-terminating VT n=36, sustained VT n=33; VT: Ventricular tachycardia, CL: cycle length.

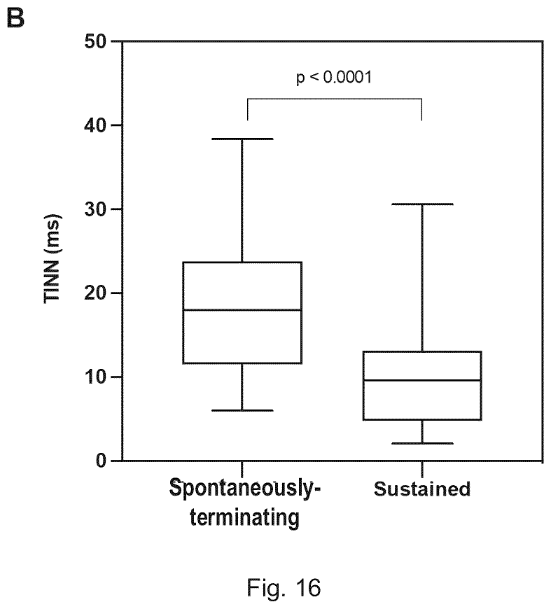

Referring to FIGS. 15, 16, and 17, spontaneously-terminating VT showed increased cycle length variation in the first 10 CLs: Spontaneously-terminating VT episodes had (FIG. 15) higher standard deviation of successive interbeat intervals, (FIG. 16) increased triangular interpolation of the NN interval histogram (TINN) and a (FIG. 17) greater time series autoregression coefficient. Episodes of spontaneously-terminating VT n=36, sustained VT n=33.

Referring to FIG. 18, 3D scatter plot showing the feature distribution: CL standard deviation, AR coefficient and TINN are plotted. There is separation of non-sustained and sustained VT CL features, with some overlap, suggesting that it is possible to use these features to distinguish spontaneously-terminating VT from sustained VT episodes. Episodes of spontaneously-terminating VT n=36, sustained VT n=33; CL: cycle length, AR: autoregression, TINN: triangular interpolation of the NN interval histogram.

Stability parameters can be used to predict whether VT episode will be sustained In order to determine whether outcome of the VT episode could be predicted using the first 10 CLs of each episode, the features above were fed into a random forest classifier. The features used in the final model were mean CL, SD, RMSSD, TINN, AR coefficient and constant. A k-fold cross validation was performed, where k=10. This resulted in a mean accuracy of 0.76 (95% CI 0.69 to 0.82), mean precision of 0.77 (95% CI 0.64 to 0.9), mean recall 0.75 (95% CI 0.66 to 0.84), mean f1 score 0.74 (95% CI 0.67 to 0.81), mean AUROC 0.76 (95% CI 0.69 to 0.83).

### Discussion

We report the first description of VT CL variability as a potential discriminator between sustained and non-sustained ventricular arrhythmia, using data from clinical VT episodes recorded on ICD devices. Our data demonstrate the potential utility of CL variability in the first 10 CLs of VT to guide ICD therapies.

Increased VT CL stability is associated with sustained tachycardia requiring therapy In this study, we found that sustained VT reaches nadir CL faster than non-sustained episodes. Additionally, increased CL variability was associated with spontaneously terminating VT. In particular, the combination of 6 features in a random forest classifier, using only the first 10 CLs of the VT, was able to predict VT termination with an accuracy of 76%. Although the present accuracy is not sufficient to withhold therapies in all relevant cases, a proportion of VT episodes with the highest CL variability could have therapies delayed to reduce unnecessary shocks, where conventional programming might deliver a therapy, particularly at lower VT rates.

Variability in Cycle Length Occurs Prior to Tachycardia Termination and is Associated with Non-Sustained Tachycardias

Our findings are in agreement with previous studies that have associated CL variability with re-entrant tachycardia termination. Callans et al studied 55 episodes of hemodynamically tolerated monomorphic VT during inpatient telemetry monitoring, ambulatory Holter monitoring or during electrophysiology studies; while Ortiz et al utilised the canine sterile pericarditis model to study atrial flutter (20, 22). Increased variability in CL was shown in both of these studies to occur just before termination.

Similarly, studies on Ibutilide, have shown that it can terminate atrial flutter with increased CL variability before termination (23). Chung, Pogwizd and colleagues studied a group of six patients with prior myocardial infarction undergoing arrhythmia surgery and found that spontaneously-terminating VT was associated with increased CL oscillations compared to sustained tachycardia and hypothesised this may be due to dispersion of refractoriness (24).

Cismaru et al describe a group of patients with ventricular fibrillation (VF) in Brugada Syndrome (BS) (25). They found spontaneously-terminating VF was associated with less CL variability than sustained VF requiring a shock. However only 5 episodes of VF in this study were of spontaneous VF, the remainder were during defibrillation threshold testing or programmed ventricular stimulation.

Our data are consistent with the above, but are novel in that they are entirely derived from clinical VT episodes from patients with ICDs.

### Mechanism for Association Between CL Variability and Termination of Re-Entrant Arrhythmias

In a canine atrial tissue model of atrial flutter, Frame and colleagues (26, 27) described that spontaneous CL oscillations were due to interval-dependent changes in conduction velocity action potential duration, which increase the likelihood of spontaneous tachycardia termination. Because of the restitution properties of myocardium, local changes in conduction occur due to the effects of the local CL and diastolic interval on the subsequent interval's conduction velocity and action potential duration, respectively (28). These oscillations then increase the chances of the action potential wavefront encountering refractory tissue, leading to conduction block and tachycardia termination. Similar findings were subsequently described in a canine pericarditis model of atrial flutter, the canine post-infarction heart (22, 29) and in atrioventricular re-entrant tachycardia in humans (30).

### Avoiding Harmful ICD Shocks

Observational data suggest that appropriate ICD shocks are associated with increased mortality (31). In particular, the finding that ATP is not associated with increased mortality reaffirms the potential harmful effects of ICD shocks beyond the effects of VT itself (31). ICD shocks reduce quality of life (8), may rarely be proarrhythmic (9), and have been associated with excess mortality (10). Potential mechanisms for increased mortality include shock induced myocardial injury or stunning (11, 32). There is therefore strong evidence for the avoidance of ICD shocks, unless absolutely necessary.

Historically, there has been a focus on avoiding “inappropriate” therapies e.g. due to SVT rather than a ventricular arrhythmia. However, more recently multiple randomised controlled trials have demonstrated the benefits of more conservative programming to reduce “unnecessary” ICD therapies for VT that are either not hemodynamically compromising or may self-terminate (13-17). Germano et al showed that ICD shocks in the major ICD randomised controlled trials out-numbered sudden cardiac deaths in the control group by a factor of 2 to 3 (33). While a proportion of these are inappropriate shocks, even appropriate shocks out-number sudden cardiac deaths in the control groups, indicating that a proportion of these shocks are potentially “unnecessary” as either the VT would not have been hemodynamically significant, would have self-terminated, or both. Methods to accurately identify the episodes that are likely to self-terminate are therefore sorely needed.

CL variability could be used to delay therapies for VT that are likely to be non-sustained Currently CL stability is often used as an SVT discriminator to reduce inappropriate shocks that may occur due to AF with a rapid ventricular response (18). Our data suggest that the CL stability or variability may be used to delay therapies for VT episodes that may spontaneously terminate and to prevent unnecessary shocks. Incorporating this feature in ICD detection algorithms has the potential to further reduce unnecessary shocks for VTs that will self-terminate. In particular, this could be incorporated into the first VT zone where tachycardia rate is likely to be lower and therefore more likely to be tolerated hemodynamically by the patient. CL variability applied to first 10 CLs (lasting approximately 3.4 s) would allow ample time to defer a therapy if appropriate. Additionally, CL variability could be combined with quantification of electromechanically coupling to identify episodes most suited for delaying therapy (34).

It should be noted that the spontaneously-terminating VT episodes in this study did not receive therapies even with existing programming algorithms and settings, so would not have benefited from additional algorithms to delay therapy. However, we postulate that a subset of episodes in our sustained group that did receive therapies, with the highest CL variability, may have self-terminated if CL variability was incorporated as an additional feature and therapy delayed, though this needs to be tested prospectively.

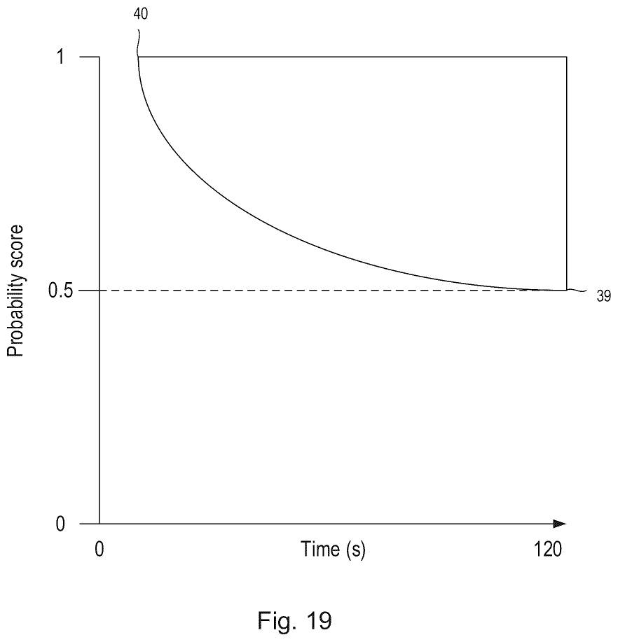

Referring to FIG. 19, a plot of the probability that the signal indicative or predictive of ventricular tachycardia is sustained (1) or spontaneously-terminating (0) for the duration of the signal being monitored is shown. A classification threshold 39 at a probability of 0.5 indicates whether the VT is sustained VT (≥0.5) or spontaneously-terminating VT (<0.5) as sustained ventricular tachycardia. There is a non-linear threshold 40 at which therapy, i.e. an output signal, is sent to the signal delivery means. Above this threshold, the output signal should be delivered, but below this threshold, a delay should implemented before delivery of the output signal. To arrive at this threshold, the cycle length variability parameters described above are combined to provide a likelihood (on a scale of 0-1) of sustained VT (vs spontaneously terminating VT) where ≥0.5 is sustained VT. The threshold at which the implantable stimulation device 2 (e.g. ICD) would deliver a therapy may be variable based on the likelihood the episode being sustained VT, may be user programmable (e.g. using the external system 3 in FIG. 1) and could be varied with episode duration. For example, early on during a suspected episode, there may be a higher threshold for delivery of therapy for VT, and the user may program the device to only deliver a therapy if the likelihood of sustained VT was high (e.g. above a high threshold). This threshold can be programmed to be reduced as the episode progresses in duration, so that the longer the duration of the episode, the more likely the device would be to deliver a therapy.

### Conclusion

Using data from clinical VT episodes recorded on ICD devices, we describe that increased VT CL variability is associated with spontaneously-terminating, and can be used to predict spontaneous VT termination. Given the harmful effects of unnecessary ICD shocks, this parameter could be incorporated into ICD algorithms to defer therapies where the VT is likely to be spontaneously-terminating.

### Modifications

It will be appreciated that various modifications may be made to the embodiments hereinbefore described. Such modifications may involve equivalent and other features which are already known in the design, manufacture and use of implantable stimulation devices, ICD devices, or pacemaker devices, systems and component parts thereof and which may be used instead of or in addition to features already described herein. Features of one embodiment may be replaced or supplemented by features of another embodiment.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1. An implantable stimulation device comprising:
signal receiving means for receiving a signal from a heart; and
signal delivery means for delivering an output signal to the heart;
in response to receiving the signal from the signal receiving means, the implantable stimulation device configured to:
determine whether the signal is indicative of ventricular tachycardia;
upon positive determination that the signal is indicative of ventricular tachycardia, determine whether the signal is indicative of sustained ventricular tachycardia or spontaneously-terminating ventricular tachycardia; and
upon positive determination that the signal is indicative of sustained ventricular tachycardia, send an output signal via the signal delivery means; else if
upon positive determination that the signal is indicative of spontaneously-terminating ventricular tachycardia, delay sending the output signal via the signal delivery means.

2. The implantable stimulation device of claim 1, wherein to determine whether the signal is indicative of ventricular tachycardia the device is configured to:
determine whether the signal is indicative of ventricular tachycardia or ventricular fibrillation.

3. The implantable stimulation device of claim 1, wherein to determine whether the signal is indicative of ventricular tachycardia, spontaneously-terminating ventricular tachycardia, sustained ventricular tachycardia, or ventricular fibrillation, the device is configured to:
analyse a characteristic of the signal; or
analyse the variance of a characteristic of the signal.

4. The implantable stimulation device of claim 3, wherein a or the variance of a characteristic of the signal determines a probability score that the signal is indicative of sustained ventricular tachycardia, and if the probability score is above a threshold, it is determined that the signal is indicative of sustained ventricular tachycardia; else if the probability score is below a threshold, it is determined that the signal is indicative of spontaneously-terminating ventricular tachycardia.

5. The implantable stimulation device of claim 4, wherein the threshold is dependent on time after the determination that the signal is indicative of ventricular tachycardia.

6. The implantable stimulation device of claim 1, wherein a or the characteristic of the signal is based on a heartbeat cycle length.

7. The implantable stimulation device of claim 1, wherein a or the characteristic of the signal is based on differences between successive heartbeat cycle lengths.

8. The implantable stimulation device of claim 1, wherein a or the characteristic of the signal is measured for at least ten repetitions.

9. The implantable stimulation device of claim 1, wherein a or the characteristic of the signal is based on one or more of:
mean heartbeat cycle length;
heartbeat cycle length standard deviation;
root mean square of successive heartbeat RR interval differences;
a triangular interpolation of the NN interval histogram;
a first order autoregression model coefficient; and
a first order autoregression model constant.

10. The implantable stimulation device of claim 3, wherein a or the characteristic of the signal is selected based on a random forest classifier to predict the probability of arrhythmia spontaneous termination.

11. The implantable stimulation device of claim 1, wherein upon positive determination that the signal is indicative of spontaneously-terminating ventricular tachycardia, the device is configured to:
monitor the signal to determine whether the signal remains indicative of spontaneously-terminating ventricular tachycardia or whether the signal is indicative of sustained ventricular tachycardia; and
upon positive determination that the signal is indicative of sustained ventricular tachycardia, send an output signal via the signal delivery means.

12. The implantable stimulation device of claim 1, wherein upon positive determination that the signal is indicative of spontaneously-terminating ventricular tachycardia, the device is configured to:
monitor the signal to determine whether the signal is indicative of spontaneously-terminating ventricular tachycardia; and
upon positive determination that the signal is indicative of spontaneously-terminating ventricular tachycardia, continue to delay sending the output signal via the signal delivery means.

13. The implantable stimulation device of claim 1, wherein the signal from the heart represents the electrical activity of the heart.

14. The implantable stimulation device of claim 1, wherein the implantable stimulation device is an implantable cardioverter defibrillator.

15. The implantable stimulation device of claim 1 further comprising:
a generator configured to generate the output signal; and
a controller configured to receive the signal from the signal receiving means, determine whether the signal is indicative of ventricular tachycardia, spontaneously-terminating ventricular tachycardia or sustained ventricular, and upon positive determination, to send a trigger signal to the generator to send the output signal to the signal delivery means.

16. The implantable stimulation device of claim 15, wherein the controller comprises one or more of:
a ventricular tachycardia detector and analyser;
a therapy circuit comprising a pacing circuit and a defibrillation circuit, each operatively connected to the ventricular tachycardia detector and analyser;
a therapy controller comprising a pacing controller and a defibrillation controller;
wherein the pacing circuit is operatively connected to the pacing controller and the defibrillation circuit is connected to the defibrillation controller; and
a controller connected to the detector, pacer, and generator.

17. The implantable stimulation device of claim 13, wherein the controller comprises one or more of:
a cardiac sensing circuit for sensing electrical signals from the heart;
a rate detector for detecting a heart rate from the signal received by the cardiac sensing circuit; and
a ventricular tachycardia detector for analysing the heart rate to determine whether the heart rate is indicative of ventricular tachycardia.

18. The implantable stimulation device of claim 13 wherein the controller comprises one or more of:
a rate variance analyser;
a rate comparator;
a cycle length analyser;
a heartbeat signal morphology analyser; and
a ventricular fibrillation detector.

19. The implantable stimulation device of claim 1 wherein the signal receiving means comprises a signal receiving lead having first and second ends, the first end configured to be implantable to the heart and receive an electrical signal from the heart, and the second end configured to be connectable to the implantable stimulation device.

20. The implantable stimulation device of claim 1 wherein the signal delivery means comprises a signal delivery lead having first and second ends, the first end configured to be implantable to the heart and to deliver the output signal to the heart and the second end configured to be connectable to the implantable stimulation device.

21. A method comprising:
receiving a signal from a heart;
determining whether the signal is indicative of ventricular tachycardia;
upon positive determination that the signal is indicative of ventricular tachycardia, determining whether the signal is indicative of sustained ventricular tachycardia or spontaneously-terminating ventricular tachycardia; and
upon positive determination that the signal is indicative of sustained ventricular tachycardia, sending the output signal to the heart; else if
upon positive determination that the signal is indicative of spontaneously-terminating ventricular tachycardia, delaying sending the output signal to the heart.

22. The method of claim 21, wherein determining whether the signal is indicative of ventricular tachycardia comprises:
determining whether the signal is indicative of ventricular tachycardia or ventricular fibrillation.

23. The method of claim 21, wherein determining whether the signal is indicative of ventricular tachycardia, spontaneously-terminating ventricular tachycardia, sustained ventricular tachycardia, or ventricular fibrillation comprises:
analysing a characteristic of the signal; or
analysing the variance of a characteristic of the signal.

24. A computer program which, when executed by at least one processor, causes the at least one processor to perform the method of:
receiving a signal from a heart;
determining whether the signal is indicative of ventricular tachycardia;
upon positive determination that the signal is indicative of ventricular tachycardia, determining whether the signal is indicative of sustained ventricular tachycardia or spontaneously-terminating ventricular tachycardia; and
upon positive determination that the signal is indicative of sustained ventricular tachycardia, sending the output signal to the heart; else if
upon positive determination that the signal is indicative of spontaneously-terminating ventricular tachycardia, delaying sending the output signal to the heart.

25. A computer program product comprising a computer-readable medium, which stores a computer program that when executed is operable to:
determine whether the signal is indicative of ventricular tachycardia;
upon positive determination that the signal is indicative of ventricular tachycardia, determine whether the signal is indicative of sustained ventricular tachycardia or spontaneously-terminating ventricular tachycardia; and
upon positive determination that the signal is indicative of sustained ventricular tachycardia, send the output signal to the heart; else if
upon positive determination that the signal is indicative of spontaneously-terminating ventricular tachycardia, delay sending the output signal to the heart.
